(19) United States
(12) Patent Application Publication
(10) Pub. No.: US 2026/0026707 A1
(43) Pub. Date: Jan. 29, 2026

(54) SYSTEMS AND METHODS FOR DETECTING PATHOLOGIC BREATHS/BREATHING PATTERNS

(71) Applicant: Children's Hospital Los Angeles, Los Angeles, CA (US)

(72) Inventors: David Ledbetter, Rockwall, TX (US); Ben Yoon, Denver, CO (US); Robinder G. Khemani, Los Angeles, CA (US); Eugene Laksana, Alhambra, CA (US); Melissa Aczon, Los Angeles, CA (US); Ishmael Obeso, Westlake Village, CA (US); Andrew Eckberg, Encinitas, CA (US)

(21) Appl. No.: 18/997,110

(22) PCT Filed: Jul. 20, 2023
(86) PCT No.: PCT/US2023/028222

§ 371 Date: Jan. 20, 2025

Related U.S. Application Data
(60) Provisional application No. 63/390,744, filed on Jul. 20, 2022.

(51) Int. Cl. A61B 5/087; A61B 5/00

(52) U.S. Cl. CPC A61B 5/087; A61B 5/7267

(57) ABSTRACT

In some embodiments, a spectral tensor technique includes the steps of generating a power spectrogram and a phase spectrogram for the breath triplet; removing high frequency bins from each spectrogram; generating a spectral image by sizing each spectrogram to a pre-determined size; and assembling the spectral images generated for each breath triplet into the spectral tensor. Spectral tensors may be utilized as input to train a pathologic breath detection model. Spectral tensors may also be utilized by a pathologic breath detection model to analyze a new waveform that may or may not include a pathologic breath/pathologic breathing pattern.

### BACKGROUND

Patients supported by mechanical ventilation are at increased risk for lung injury, neuromuscular weakness, and long-term neurocognitive injury. Ventilator management has evolved to emphasize the recognition of pathologic breathing patterns when the ventilator may not be meeting the patient's respiratory needs, notably high work of breathing and patient-ventilator dyssynchrony (PVD) (also known as patient-ventilator asynchrony (PVA)). Both pathologies are common, associated with adverse clinical outcomes, and challenging to recognize at the patient's bedside without specialized equipment and the expertise to interpret them.

### SUMMARY

In some embodiments, a method of creating a pathologic breathing detection model includes: obtaining, by a computing device, a spectral tensor, wherein the spectral tensor is generated by: generating a power spectrogram and a phase spectrogram for a breath triplet of a training waveform, wherein the training waveform is a flow waveform, an airway pressure waveform, and/or an esophageal manometry waveform: removing high frequency bins from each spectrogram: generating a spectral image by sizing each spectrogram to a pre-determined size: assembling the spectral images generated for each breath triplet into the spectral tensor; and training a machine learning model to detect a pathologic breath and/or pathologic breathing pattern in a waveform using the spectral tensor as a training input.

Embodiments further include a computer-implemented analysis method that includes: obtaining, by the computer, a new waveform, the new waveform being either a flow waveform and/or an airway pressure waveform; and evaluating the new waveform using a pathologic breath detection model to detect a pathologic breath and/or pathologic breathing pattern in the new waveform, wherein the pathologic breath detection model was trained using a spectral tensor as input, each spectral tensor generated from a breath triplet of a training waveform, and wherein the training waveform is a flow waveform, an airway pressure waveform, and/or an esophageal manometry waveform. The spectral tensor is generated by a spectral tensor technique comprising the steps of: generating a power spectrogram and a phase spectrogram for the breath triplet; removing high frequency bins from each spectrogram; generating a spectral image by sizing each spectrogram to a pre-determined size; and assembling the spectral images generated for each breath triplet into the spectral tensor.

Embodiments include a computer program product that includes a non-transitory computer readable medium having embodied thereon a computer program comprising computer code, the code including: code for a pathologic breath detection model to detect a pathologic breath and/or pathologic breathing pattern in a waveform, wherein the waveform is a flow waveform and/or an airway pressure waveform. Wherein the pathologic breath detection model was trained with a spectral tensor generated by a method comprising: generating a power spectrogram and a phase spectrogram for a breath triplet in a training waveform, wherein the training waveform is a flow waveform, an airway pressure waveform, and/or an esophageal manometry waveform; removing high frequency bins from each spectrogram; generating a spectral image by sizing each spectrogram to a pre-determined size; and assembling the spectral images generated for each breath triplet into the spectral tensor.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 illustrates a block diagram of a system to develop/train a pathologic breathing detection (PBD) model from a machine learning model (an untrained PBD model), according to some embodiments.

FIG. 2 illustrates a flowchart of a method of generating inputs to train the machine learning model to detect a pathologic breath and/or pathologic breathing pattern in a waveform, according to some embodiments.

FIG. 3 illustrates a graph of an exemplary annotated waveform that includes a previous (left), current (middle), and subsequent breath (right), according to some embodiments.

FIG. 4 illustrates a graph of annotated waveforms, according to some embodiments. The three waveforms represent the flow (top), airway pressure (middle), and esophageal manometry (bottom). Upside triangles indicate inspiration markers, while downside triangles indicate expiration markers. In this example, periods with a pathologic breath/pathologic breathing pattern and/or artifacts are highlighted by red shaded boxes.

FIG. 5 illustrates a flowchart of a method of determining respiratory effort, according to some embodiments.

FIG. 6 illustrates a graphical depiction of calculating respiratory effort ΔP_{ES} from an esophageal manometry waveform (blue curve), according to some embodiments.

FIG. 7 illustrates a flowchart of a spectral tensor technique to generate spectral tensors from breath triplets in waveform data, according to some embodiments.

FIG. 8 illustrates a graph of a wave to illustrate terminology utilized to characterize a wave, according to some embodiments.

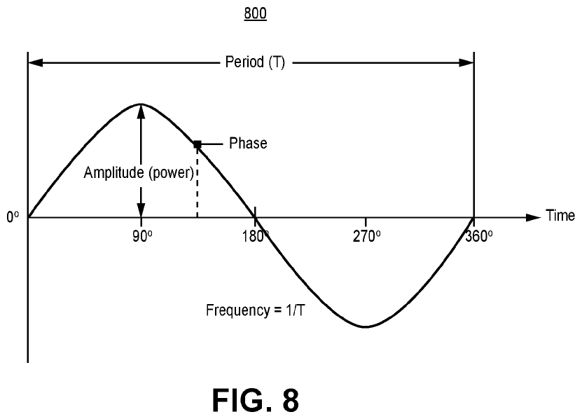

FIG. 9 illustrates the correlation between increasing and decreasing flow of a breath to a flow power spectral density spectrogram and a flow phase spectrogram, according to some embodiments.

FIG. 10 illustrates a graph showing the application of a Fourier transform window to a waveform, according to some embodiments.

FIG. 11 illustrates an example power spectral density (PSD) spectrogram and phase spectrogram generated utilizing a Fourier transform, according to some embodiments.

FIG. 12 illustrates an example of a power spectral density spectrogram (PSD) and a phase spectrogram generated by a step of a spectral tensor technique, according to some embodiments.

FIG. 13 illustrates a fixed-length window applied to a spectrogram to produce a desired fixed-size image—a spectral image having the size of the window, according to some embodiments.

FIG. 14 illustrates an example of a 4-channel spectral tensor, according to some embodiments.

FIG. 15 is a block diagram of system to identify/diagnose a pathologic breath and/or pathologic breathing pattern utilizing a trained PBD model, according to some embodiments.

FIG. 16 illustrates a flow chart of a method to detect a pathologic breath and/or pathologic breathing pattern in a waveform using a trained PBD model, according to some embodiments.

FIG. 17 illustrates graphs of a receiver operating characteristic curve and number needed to alert (NNA) as a function of missed detection rate for a PBD model trained to detect binary Double Cycle (DC) breaths, according to some embodiments.

FIG. 18 illustrates graphs of a receiver operating characteristic curve and number needed to alert (NNA) as a function of missed detection rate for a PBD model trained to detect Reverse Trigger and Inadequate Support as underlying dyssynchrony types (a multi-target PBD model), according to some embodiments.

FIG. 19 illustrates a graph of a receiver operating characteristic curve of for a PBD model trained to detect high respiratory effort (ΔP_{ES}≥20 mmHg), according to some embodiments.

FIG. 20 illustrates graphs comparing true/actual values of ΔP_{ES} and predictions for two different patients generated by a regression PBD model trained to predict high respiratory effort, according to some embodiments.

FIG. 21 illustrate graphs of flow and airway pressure waveforms and corresponding spectrograms of breath triplets to compare normal central breaths with dyssynchronous central breaths, according to some embodiments.

### DETAILED DESCRIPTION

A ventilator not meeting a patient's respiratory needs can result in pathologic breathing patterns, including patient-ventilator dyssynchrony (PVD) or asynchrony (PVA) and high work of breathing/high respiratory effort, that are associated with adverse clinical outcomes. Ventilator asynchronies pose a risk of injury to a patient's lung while mechanically ventilated. Types of dyssynchrony include: reverse trigger where a negative drop in esophageal manometry, that exceeds 2 cm H₂O, occurs during the inspiratory phase of a mandatory time cycled breath; flow undershoot/starvation where airway pressure has a concave rising limb during flow decelerating pattern or concave flow waveform and esophageal manometry has a continued negative trajectory during inspiratory flow; premature termination/cycling where flow has a sharp decrease in expiratory flow at the end of the breath followed by a rebound increase and gradual decrease to baseline with concomitant airway pressure depression at the end of inspiration below baseline, and esophageal manometry continues to have a negative deflection at end of the ventilator delivered breath: and inadequate support where the ventilator prematurely terminated breath (premature termination) or did not give enough airflow (flow undershoot).

One of the most injurious consequences of PVD are double cycled (DC) breaths when two breaths are delivered instead of one. There are a number of underlying asynchronies or dyssynchrony subtypes that can lead to DC breaths. For example, reverse trigger and inadequate support are two underlying PVD mechanisms of DC breaths. Because treatment strategies differ, it is important to differentiate these asynchronies and subtypes of asynchronies from one another. Identifying an abnormality in the ventilator waveforms is difficult and typically requires a clinician with specialized training at the bedside. Because this type of training requires a large investment—both in time and money—not every hospital/care setting has clinicians with this specialized training. Additionally, access to esophageal manometry data, which is the gold standard for patient respiratory effort, is rarely available. Therefore, there is a need for an autonomous detection technique to diagnose/treat pathologic breathing, e.g., PVD/PVA, and high respiratory effort, that does not require a clinician with specialized training to analyze the waveforms and/or esophageal manometry data. Additionally, it has been found that about 80% of children experience asynchrony. Waveforms of children ranging from babies to teenagers are different from adult waveforms, so there is a need for an automatic detection technique that can diagnose/treat pathologic breathing in a pediatric population. For these reasons, implementations described herein improve the delivery of care with the use of a computerized process to perform tasks or roles that were not previously performed or previously could only be performed by clinicians with specialized training and/or improve the delivery of care to pediatric patients.

In some implementations, a machine learning model is trained using three channels of waveform data to generate a pathologic breathing detection (PBD) model. For example, the three channels of waveform data are flow, airway pressure, and esophageal manometry. Spirometry flow is an indication of how much air is moving back and forth/in and out. Airway pressure is an indication of how much force is being utilized to move air. Esophageal manometry utilizes a tube that extends through the nose down through the esophagus and provides an indication of the patient effort to breath. Using information/data obtained from three channels of waveform data may improve the accuracy and/or specificity of the PBD model. For example, esophageal manometry data may be used as “truth” in the training of the machine learning model.

In some implementations, a machine learning model is trained using two channels of waveform data to generate a PBD model. For example, the two channels of waveform data are flow and airway pressure. Using two channels of waveform data may improve the accuracy and/or specificity of a PBD model that uses two channels of unseen waveform data as input. Further, training a PBD model using only these two channels yields a PBD model that may be utilized when esophageal manometry is not available.

In some implementations, a machine learning model is trained using waveform data from a pediatric population to generate a PBD model. Using pediatric waveforms to train a machine learning model may improve the accuracy and/or specificity of the PBD model to detect pathologic breathing patterns in pediatric patients.

In some implementation, a machine learning model is trained using waveform data from an adult population to generate a PBD model. Using adult waveforms to train a machine learning model may improve the accuracy and/or specificity of the PBD model to detect pathologic breathing patterns in adult patients.

In some implementations, a spectral tensor creation technique is utilized to generate inputs to train a machine learning model to generate a PBD model. For example, the spectral tensor creation technique may transform a waveform (e.g., waveforms relevant to respiration including flow and airway pressure waveforms) into spectrograms that better represent an underlying problem to a machine learning model, resulting in improved model accuracy on unseen data. One metric that may be used to characterize the performance of a machine learning model is the area under the receiver operating characteristic curve (AUROC). In one example, a model trained with flow and airway pressure spectrograms had an AUROC of 0.984 whereas models trained with raw flow and airway pressure waveforms had AUROCs of 0.655 and 0.811, respectively, for the same test set (not shown).

In some implementations, the spectral tensor technique filters high frequency data from generated spectrograms. This may improve the accuracy of the trained machine model by enabling the model to be more robust than unprocessed models to noisy data.

In some implementations, the spectral tensor technique may transform spectrograms into spectral images. For example, the spectral images may have a predetermined size. This may improve the training process by training the PBD model with spectral images that have a consistent size.

In some implementations, the PBD model uses two channels of waveforms as input to identify/detect pathologic breathing. As one non-limiting example, the PBD model uses flow and airway pressure waveforms to identify/detect pathologic breathing. The new flow and airway pressure waveforms may or may not include an asynchrony and/or high respiratory effort. In some implementations, the flow and airway pressure waveforms may be generated by a ventilator. This may improve delivery of care by utilizing waveforms generated by equipment commonly used in a care setting.

In some implementations, the PBD model may be utilized instead of esophageal manometry. For example, the PBD model may classify a breath as either high work of breathing/high respiratory effort or low work of breathing/low respiratory effort and/or predict the respiratory effort ΔP_{ES}, the difference between the baseline P_{ES} and the minimum P_{ES}, for a breath. This may improve delivery of care in locations that lack esophageal manometry.

### System to Develop/Train a PBD Model

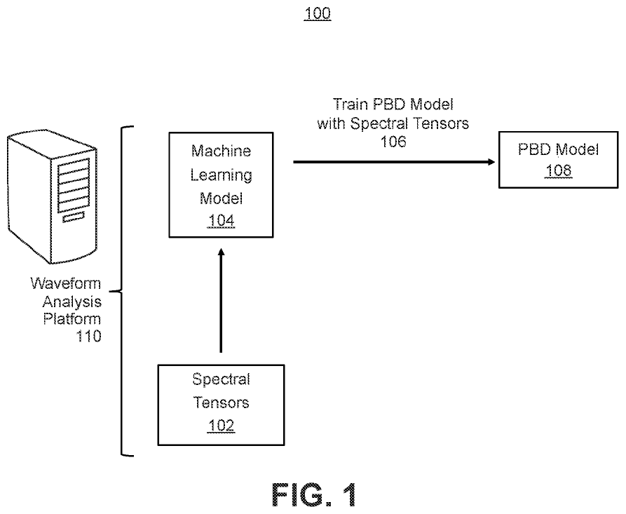

FIG. 1 illustrates a block diagram of a system 100 to develop/train 106 a PBD model 108 from a machine learning model 104 (an untrained PBD model), according to some embodiments. In some implementations, the PBD model 108 detects at least one type/subtype of asynchrony. For example, in some embodiments, the PBD model 108 is a binary DC breath detection model. i.e., identify double-cycled breaths. In one non-limiting example, a binary PBD model 108 provides a yes/no answer by identifying only the breaths identified as being a pathologic breath. In other embodiments, the PBD model 108 is a multi-target dyssynchrony detection model trained to detect a plurality of types/subtypes of asynchrony. In one example, the multi-target dyssynchrony detection model detects/identifies reverse trigger and inadequate support. In additional embodiments, the PBD model 108 is a respiratory effort detection model. In some implementations, the PBD model 108 is a binary respiratory effort detection model that classifies each breath as either high work of breathing/high respiratory effort or low work of breathing/low respiratory effort. In other implementations, the PBD model 108 is a regression respiratory effort model that predicts the actual values of respiratory effort ΔP_{ES} which is the difference between the baseline P_{ES} and the minimum P_{ES}.

The system 100 includes a waveform analysis platform 110 with a machine learning model 104—an untrained PBD model. The waveform analysis platform 110 includes at least one computer comprising a computer readable medium and a processor. In some implementations, the machine learning model 104 is stored on the computer readable medium. Instructions stored on the computer readable medium may be executed by the processor of the waveform analysis platform 110. In some implementations, the machine learning model 104 is a deep convolutional neural network (CNN). In one non-limiting example, the CNN may be developed using the PyTorch library, a binary cross-entropy (BCE) for the loss function, and an AdaBelief optimizer.

A plurality of spectral tensors 102 are utilized as input to the machine learning model 104 to generate a PBD model 108. The spectral tensor technique 700 discussed below may be utilized to generate the spectral tensors 102. In some implementations, one computer of the waveform analysis platform 110 generates the spectral tensors 102 and trains 106 the machine learning model 104. In other implementations, more than one computer of the waveform analysis platform 110 is utilized to train the machine learning model 104. For example, a first computer may generate the spectral tensors and a second computer may train 106 the machine learning model 104 using the spectral tensors 102.

The spectral tensors 102 are grouped into a training set, a validation set, and a test set, which are used to develop 106 the machine learning model 104. The training set is utilized for initial training of the machine learning model 104. The validation set is utilized to validate results of the PBD model 108 and/or to provide additional training for the machine learning model 104 and information for the spectral tensors 102. For example, model training hyperparameters such as the loss function, optimizer and learning rate, and spectral image parameters such Fourier window size and spectrogram size, may be optimized with the validation set. The test set is utilized to test operation of the PBD model 108.

In some implementations, input to train the machine learning model 104 further includes at least one dataset. As discussed below in greater detail, an input data set may be generated by method 200 and/or method 500.

### Generating Input for the Machine Learning Model

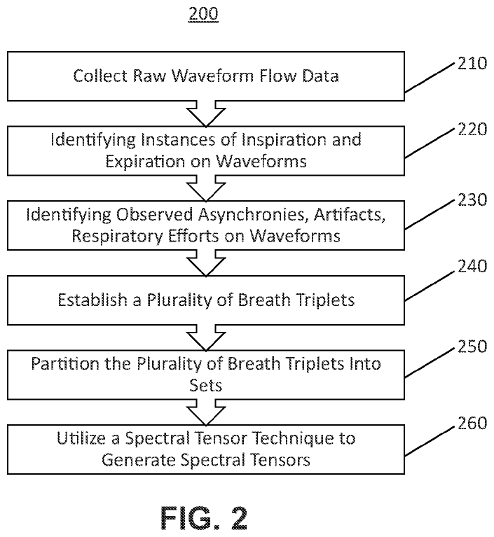

FIG. 2 is a flowchart of a method 200 to generate inputs that may be utilized to train 106 a machine learning model 104 to detect a pathologic breath pattern from waveform according to some embodiments of this disclosure. At Step 210, a waveform is collected. In some implementations, the waveform collected for training purposes may be a flow spirometry waveform and/or an airway pressure waveform. In other implementations, the waveform collected for training purposes includes a flow spirometry waveform, an airway pressure waveform, and/or an esophageal manometry waveforms. The esophageal manometry waveform may be used as the target truth for training 106 of the machine learning model 104.

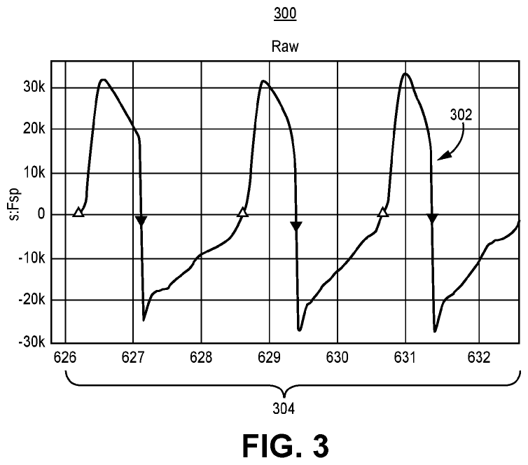

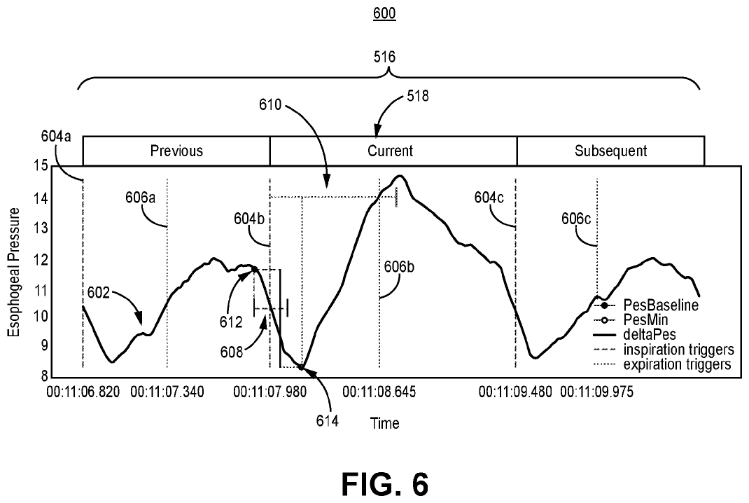

At Step 220, instances of inspiration and expiration are identified. Identifying instances of inspiration and expiration may include annotating or labelling the waveform and/or collating information about inspiration and expiration into a dataset. In some embodiments, a software program is used to annotate the waveforms with inspiration and expiration markers. In one implementation, breaths are delineated by a flow crossing algorithm. FIG. 3 illustrates a graph 300 of an exemplary annotated waveform 302. In this example, upside triangles are used as inspiration markers and downside triangles are used as expiration markers. As another example, a marker for inspiration and/or expiration may be a vertical line (see e.g., FIG. 6). In some implementations, an individual breath is defined to start at one inspiration marker and end at the time step immediately preceding the next inspiration marker. The time for each inspiration/expiration is an example of information that may be collated into a dataset.

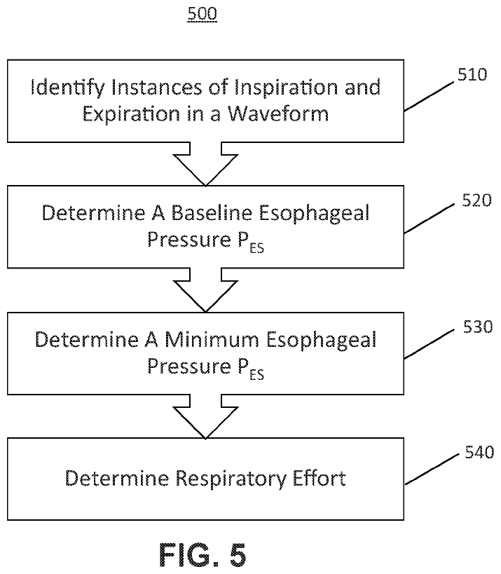

At Step 230, observed asynchronies and/or artifacts in the waveforms are identified. Identifying asynchronies and/or artifacts may include annotating or labelling the waveform and/or collating information about the asynchrony and/or artifact into a dataset. Step 230 may further include determining a respiratory effort. As a non-limiting example, Step 230 may include the execution of Steps 520-540 of method 500 which is discussed below with reference to FIGS. 5-6. The respiratory effort may be annotated/labeled on the waveform and/or included in a dataset. Annotation of a respiratory effort may include determining a baseline P_{ES} and a minimum P_{ES} from the esophageal manometry waveform, calculating ΔP_{ES}, and/or identifying a breath as a high respiratory effort or a low respiratory effort.

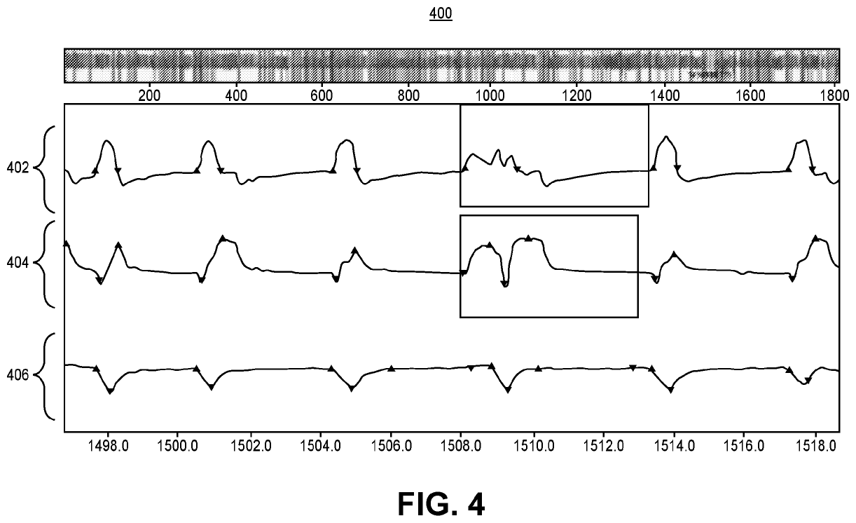

In some implementations, Step 220 and Step 230 produce annotated flow waveforms and/or airway pressure waveforms. For example, the waveforms may be annotated with observed asynchronies and/or artifacts. The waveforms may be further annotated with respiratory effort. FIG. 4 illustrates a graph 400 of an annotated flow waveform 402 and an annotated airway pressure waveform 404, according to some embodiments. In some implementations, annotation of asynchronies and/or artifacts on the waveforms is conducted manually. For example, at least one person with specialized training in clinical ventilator waveform interpretation may manually review and annotate the waveform. In some implementations, the esophageal manometry waveform 406 is utilized as an aid in the classification/annotation of asynchronies and/or respiratory efforts in the flow and airway pressure waveforms 402, 404. In some implementations, periods with dyssynchronies or artifacts are highlighted or indicated by a box and/or the annotation. In graph 400, a shaded box highlights a period that has been annotated. A highlighted period may include one or more breaths. Types/subtypes of dyssynchrony that may be annotated on the waveform include reverse trigger, flow undershoot/starvation, premature termination/cycling, and/or inadequate support. In this example, upside triangles are used as inspiration markers, downside triangles are used as expiration markers, red shaded boxes are used to highlight periods with dyssynchronies or artifacts, and the type of dyssynchrony in the highlighted period is labeled. Each breath may have more than one label/annotation of a dyssynchrony, an artifact—in other words more than one dyssynchrony or artifact may be identified for a breath. As discussed below in greater detail, the annotations of each breath are utilized to classify breath triplets.

Returning to method 200, in some implementations Step 230 is executed before Step 220. In other implementations, Steps 220 and 230 are executed simultaneously.

At Step 240, a breath triplet is established. A breath triplet is a group of three consecutive breaths. Optionally, the inspiration and expiration markers are utilized to delineate a breath triplet in the waveform. A waveform typically includes a plurality of breath triplets. The waveform 302 illustrated in FIG. 3 has one breath triplet 304. A breath triplet 304 includes a previous breath (left), a current breath (middle), and a subsequent breath (right). In some implementations, the breath triplets 304 are generated/identified from the flow waveforms and airway pressure waveforms. In other implementations, the breath triplets 304 are generated from flow waveforms, airway pressure waveforms, and esophageal manometry waveforms.

Each breath triplet 304 is classified based on the identifications generated by Step 230. Breaths that are not in a highlighted period or not identified as having an asynchrony/artifact/high respiratory effort are classified as “normal.” Classification of each breath triplet 304 is based on the middle breath. Thus, each breath triplet 304 inherits all the labels associated with the middle breath, with the left and right breaths in the breath triplet 304 (previous and subsequent breaths, respectively) providing temporal context for the middle breath during classification. Breath triplets 304 that may be excluded from the plurality of breath triplets include any breath triplet 304 containing a breath greater than ten (10) seconds, any breath triplet 304 containing a breath less than 0.25 seconds, and/or any breath triplet 304 with a “normal” middle breath but a dyssynchronous left or right breath. In at least one implementation, the dataset(s) containing the collated information/data from Step 220 and/or Step 230 may be utilized to classify breath triplets.

Steps 210, 220, 230, and 240 may be described as a method of generating breath triplets.

At Step 250, the breath triplets 304 are randomly partitioned into training, validation, and test sets. In some implementations only the spirometry flow and airway pressure breath triplets are randomly partitioned into training, validation, and test sets. In other implementations, spirometry flow, airway pressure, and esophageal manometry breath triplets are randomly partitioned into training, validation, and test sets. In one example, all the breath triplets 304 from a single person belong to only one of these sets.

In some implementations, at least some of the information/data identified or generated by Steps 220-250 is collated into at least one dataset. Information/data generated by method 200 may include subject ID, waveform type (flow, airway pressure, esophageal manometry), time of inspiration/expiration, breath length, time frame (start-stop) for an pathologic breath, pathologic breath pattern or an artifact, bP_{ES}, mP_{ES}, ΔP_{ES}, identifying a breath as a high respiratory effort or a low respiratory effort, breath triplets, and/or which set (training, validation, test) a breath triplet is assigned to.

At Step 260, a spectral tensor technique 700 is utilized to generate spectral tensors of the breath triplets in the training, validation, and test sets. As illustrated in FIG. 1, the waveform analysis platform utilizes the spectral tensors 102 to train the PBD model 104. In some implementations, at least one dataset generated by method 200 and/or method 500 is utilized as an input to train the PBD model 104.

The trained PBD model 108 may detect/classify a single type of pathologic breathing (i.e., a single target PBD model 108); detect/classify multiple types of pathologic breathing (i.e., a multi-target PBD model 108); and/or predict an actual value for respiratory effort ΔP_{ES} (i.e., a regression PBD model 108). In one implementation of a single target PBD model 108, the PDB model 108 is a binary Double Cycle (DC) breath detection model. In another implementation of a single target PBD model, the PDB model is a breathing classification model, e.g., a binary respiratory model that classifies each breath as either a high respiratory effort or a low respiratory breath effort. In an implementation of a regression PBD model, the regression PBD model 108 is a regression model that predicts the actual value of a respiratory effort ΔP_{ES}. The predictions generated by the regression PBD model 108 may be used to classify a breath as either a high respiratory effort or a low respiratory breath effort.

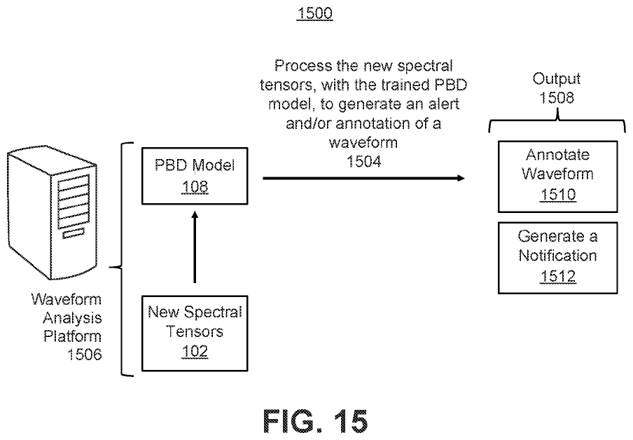

As discussed below in reference to FIG. 15, the trained PBD model 108 may be incorporated as a piece of software which may be uploaded to a waveform analysis platform 1506. In some implementations, a waveform analysis platform 1506 may include one or more trained PBD models 108.

### Determining Respiratory Effort

FIG. 5 is a flowchart of a method 500 to determine respiratory effort and FIG. 6 illustrates a graphical depiction 600 of calculating a respiratory effort, from an esophageal manometry waveform 602 (blue curve), according to some embodiments. In at least one implementation, the result of method 500 is utilized to classify a breath. In some implementations, a waveform is annotated with the result of method 500. As discussed above, Steps 520-540 may be incorporated into method 200. In other implementations, information/data generated by method 500 may be collated into at least one dataset.

At Step 510, instances of inspiration and expiration in an esophageal manometry waveform are identified. Identifying inspiration and expiration may include annotating or labelling the waveform and/or collating information about inspiration and expiration into a dataset. In some implementations, vertical lines are used to identify/annotate instances of inspiration and expiration in the waveform. For example, as illustrated in graph 600 of FIG. 6, vertical green dashed lines 604 are utilized to mark inspiration triggers and vertical red dashed lines 606 are utilized to mark expiration triggers of a breath triplet 516. In method 200, Step 510 is incorporated into Step 220.

At Step 520 a baseline esophageal pressure (bP_{ES}) is determined for the current breath 518 of a breath triplet 516. In some implementations, a window centered around the current breath's inspiration trigger (baseline window) is utilized to determine the bP_{ES}. As one non-limiting example, the baseline window 608 is a 200 ms window centered around the current breath's inspiration trigger 604b (represented by the lower horizontal dashed line bounded by vertical solid lines in FIG. 6). In other implementations, the baseline window 608 may be a 100 ms to 400 ms window. The bP_{ES}, point 612 in FIG. 6, is the maximum esophageal pressure in the baseline window 608.

At Step 530, a minimum esophageal pressure (mP_{ES}) is determined for the current breath 518 of a breath triplet 516. In some implementations, a window that extends forward from the current breath's inspiration trigger to a predetermined amount of time after the current breath's expiration trigger (a minimum window) is utilized to determine the mP_{ES}. As one non-limiting example, the minimum window 610 is a window that extends from the current breath inspiration trigger 604b to 300 ms after the current breath expiration trigger 606b (represented by the upper horizontal dashed line bounded by vertical solid lines in FIG. 6). In other implementations, the length of the extension after the expiration trigger 606b may be 100 ms to 500 ms. In the example illustrated in FIG. 6, the minimum window 610 has a time extent less than the time between inspiration trigger 604b and inspiration trigger 604c. Also, as illustrated in FIG. 6, the baseline window 610 extends to a time point corresponding to a peak in the esophageal pressure of the current breath 518. The mP_{ES}, point 614 in FIG. 6, is the minimum pressure in the minimum window 610.

In some implementations, Step 530 is executed before Step 520. In other implementations, the baseline window 608 and the minimum window 610 are established before the bP_{ES} and the mP_{ES} are determined/identified.

At Step 540 the respiratory effort ΔP_{ES} is classified as a low respiratory effort or a high respiratory effort. ΔP_{ES} is the difference between bP_{ES} and mP_{ES}. A low respiratory effort corresponds to a calculated respiratory effort ΔP_{ES} that is less than a threshold value while a high respiratory effort corresponds to a calculated respiratory effort ΔP_{ES} that is greater than or equal to a threshold value. As one non-limiting example, the threshold value may be clinically determined. In some implementations, the threshold value is 20 mmHg—in other words, a ΔP_{ES}<20 mmHg is classified as low respiratory effort and a ΔP_{ES}≥20 mmHg is classified as high respiratory effort. Higher values of ΔP_{ES} denote higher respiratory effort.

In some implementations, at least some of the information/data identified or generated by method 500 is collated into at least one dataset. Information/data generated by method 500 may include subject ID, time of inspiration/expiration, breath length, bP_{ES}, mP_{ES}, ΔP_{ES}, and/or identifying a breath as a high respiratory effort or a low respiratory effort.

### Spectral Tensor Technique—Generating Spectral Tensors

In at least one implementation, a spectral tensor method includes generating a power spectrogram and a phase spectrogram for a breath triplet waveform; removing high frequency bins from each spectrogram; generating a spectral image by sizing each spectrogram to a pre-determined size; and assembling the spectral images generated for each breath triplet into a spectral tensor. In some implementations, the waveform is a flow waveform and/or an airway pressure waveform. In other implementations, the waveform is a flow waveform. an airway pressure waveform, and/or an esophageal manometry waveform.

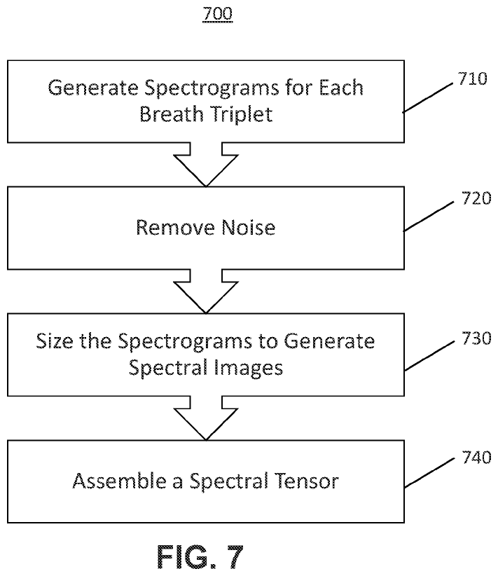

FIG. 7 is a flowchart of a spectral tensor technique 700 to generate spectral tensors 102 from breath triplets in waveform data, according to some embodiments. The waveform may be raw or annotated. Spectral tensors 102 may be utilized as input to train a PBD model 104 or as input to a PBD model 108. For example, as discussed above in reference to Step 260, spectral tensors 102 generated from the selected flow and airway pressure waveforms are utilized to train the machine learning model 104 to detect a pathologic breathing pattern.

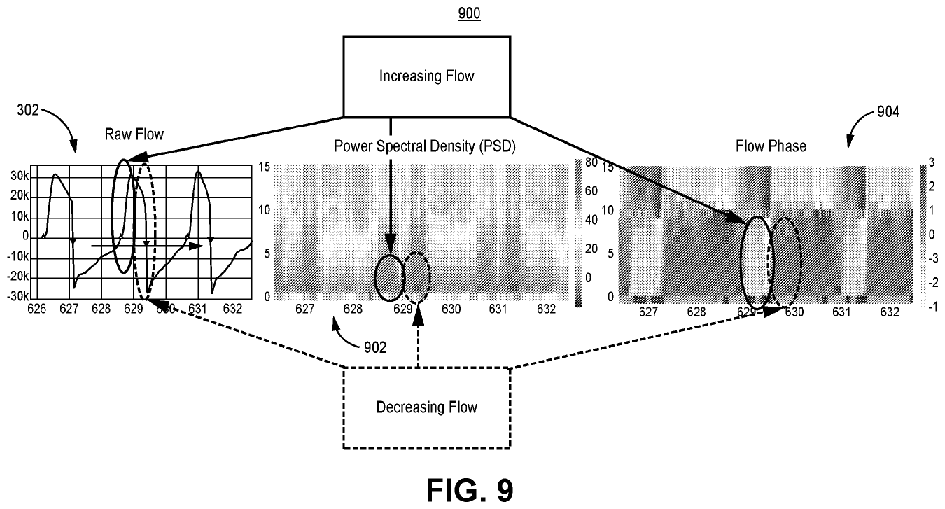

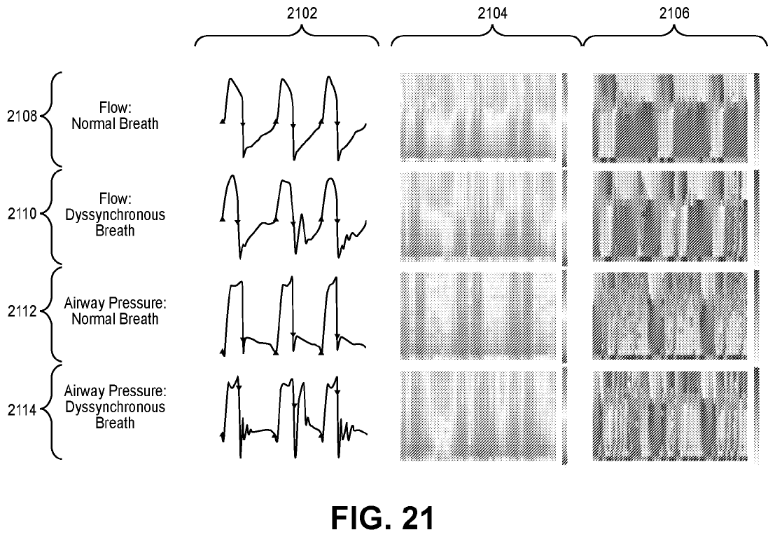

At Step 710, spectrograms are generated for each breath triplet 304. In other words, the waveform data is transformed into spectrograms. Spectrograms enable visualization of how signal strength at various frequencies changes over time. Graph 800 illustrates terminology that may be utilized to characterize a wave. A power spectral density spectrogram (PSD) and a phase spectrogram may be generated for each breath triplet. The graphs provided in FIG. 9 illustrate how the increasing and decreasing portions of flow of a breath in waveform 302 are represented in the flow power spectral density spectrogram 902 and a flow phase spectrogram 904. FIG. 21 provides examples of flow power spectral density spectrograms 2104 and flow phase spectrograms 2106 generated for breath triplets 2102 of a normal breath flow waveform 2108, a dyssynchronous breath flow waveform 2110, a normal breath air pressure waveform 2112, and a dyssynchronous breath air pressure waveform 2114. As can be seen, differences between normal and dyssynchronous waveforms are reflected in the spectrograms.

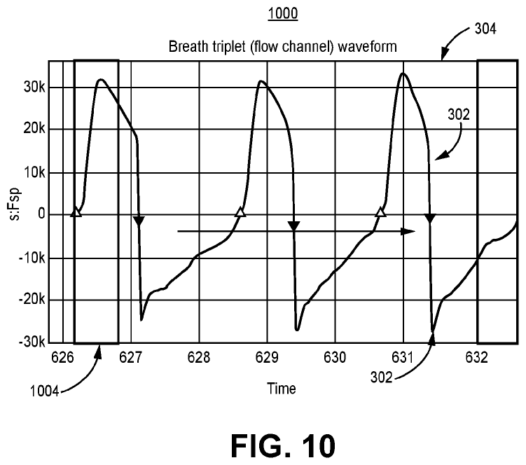

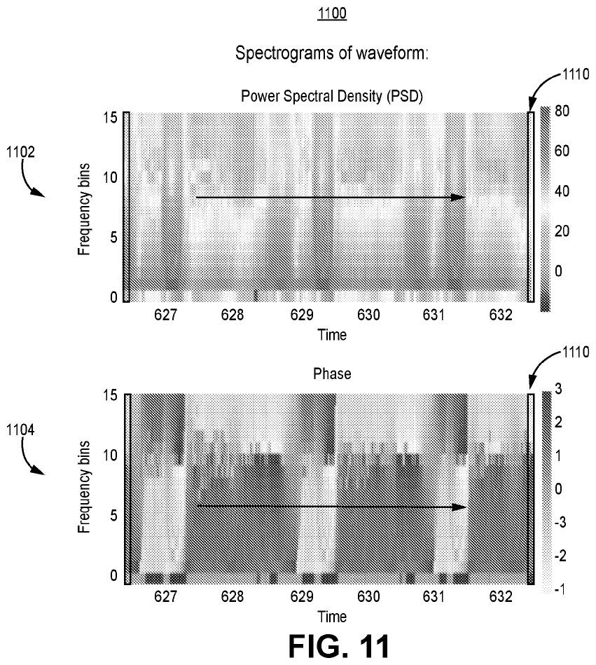

In some implementations, a Fourier transformation is utilized to generate a spectrogram from the waveform 302. For example, to generate a single spectral column of a spectrogram, a Fourier transformation is taken of a window 1004 of a waveform 302. FIG. 10 illustrates a graph 1000 showing the application of a window 1004 to a waveform 302, according to some embodiments. The window 1004 may cover only a portion of the breath triplet 304. In one non-limiting example, a Fourier extent (the window size over which the Fourier transform was performed) of 64 measurements (0.32 seconds when the waveform sampling rate is 200 Hz), and a Tukey (tapered cosine) window with 10% of the window inside the cosine tapered region is used to generate one spectral column of a spectrogram from a waveform 302. In another non-limiting example, the Fourier extent to generate a spectral column is 128 measurements. The window 1004 may be shifted over the waveform 302 data to generate subsequent spectral columns across time. In one non-limiting example, the window 1004 shifts at a stride of two—in other words the Fourier window is shifted two samples at a time to generate subsequent spectral columns of the spectrogram. FIG. 11 illustrates an example PSD spectrogram 1102 and phase spectrogram 1104 generated utilizing a Fourier transform. Each spectrogram 1102, 1104 comprises a plurality of spectral columns 1110.

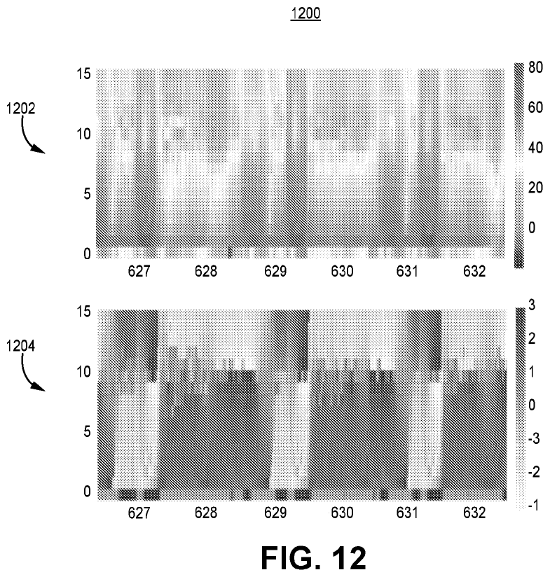

At Step 720, noise is removed from the spectrograms 1102, 1104. In one implementation, a low pass filter is used to remove noise. For example, the low pass filter may remove the high frequency bins from the spectrograms 1102, 1104. The number of frequency bins remaining after the low pass filter may be 8 to 32 frequency bins. In one non-limiting example, sixteen frequency bins remain after the high frequency bins are removed. In some embodiments, the sixteen frequency bins represent 0 to 47 Hz. FIG. 12 illustrates an example of a power spectral density spectrogram 1202 and a phase spectrogram 1204 generated by Step 720.

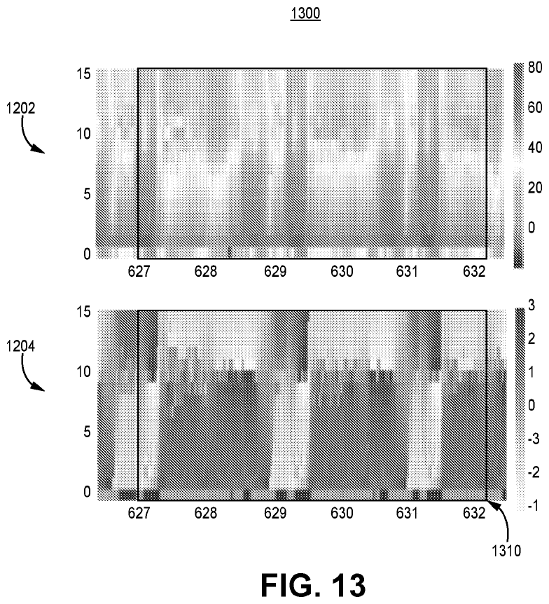

At Step 730, each spectrogram 1202, 1204 is sized to generate a spectral image with a predetermined size. If the spectrogram 1202, 1204 is smaller than a predetermined size, padding is applied to generate a spectral image. For example, zero padding may be applied. If the spectrogram 1202, 1204 has the predetermined size, no modification is required—i.e., the spectrogram is the spectral image. If the spectrogram 1202, 1204 is larger than a predetermined size, the spectrogram 1202, 1004 is cropped. For example, as illustrated in FIG. 13, a fixed-length window 1310 may be applied to each spectrogram 1202, 1204 to produce a desired fixed-size image—a spectral image having the size of the window. The size of the window 1310 may correspond to a desired number of spectral columns 1110. In one non-limiting example, the predetermined size corresponds to nine hundred (900) spectral columns 1110. In some implementations, nine hundred (900) spectral columns 1110 may correspond to a 9.3 second window. In one aspect, a 9.3 second window is long enough to cover 83% of all breath triplets.

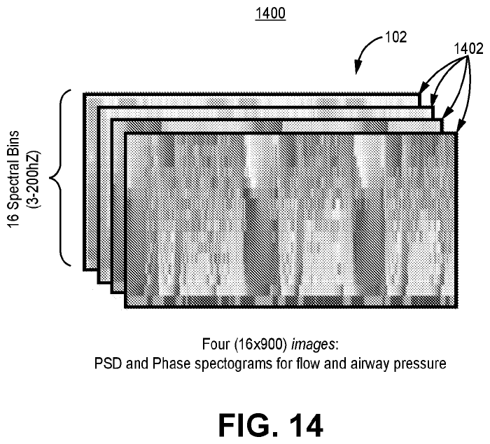

At Step 740, a spectral tensor 102 is generated by assembling a stack of spectral images 1402 of a breath triplet 304. In some embodiments, a spectral tensor 102 of a breath triplet 304 includes PSD and phase images for the flow and airway pressure waveforms. This type of spectral tensor 102 may be described as a 4-channel image. FIG. 14 illustrates an example of a 4-channel spectral tensor 102 generated by Step 740. The spectral tensors 102 may be used as input to train the PBD model 104 or as input to a PBD model 108. It was observed that the performance of a PBD model 104 trained by spectral tensors 102 generated from spectral images having nine hundred (900) spectral columns was better than, or comparable to, the performance of a PBD model 104 trained by spectral tensors 102 generated from spectral images having greater or fewer than nine hundred (900) spectral columns (not shown). It was also observed that input spectrograms containing only the lower 16 frequency bins (high frequency bins removed) resulted in better performance than input spectrograms containing all 32 frequency bins—0.997 versus 0.984 in AUROCs (not shown).

### Detecting a Pathologic Breathing Pattern With a Trained PBD Model

FIG. 15 is a block diagram of system 1500 to identify/diagnose a pathologic breath/breathing pattern utilizing a trained PBD model 108. The system 1500 includes a waveform analysis platform 1506. The waveform analysis platform 1506 may include a device with a non-transitory computer readable medium, such as but not limited to a ventilator and/or a computer. Instructions stored on the computer readable medium may be executed by a processor of the waveform analysis platform 110. In at least one embodiment, the waveform analysis platform 1506 may be positioned at a patient's bedside. In other implementations, the waveform analysis platform 1506 may be hosted in a cloud computing environment.

At least one PBD model 108 is stored on the computer readable medium. The PBD model 108 analyzes the new spectral tensors 102, generated from new flow and airway pressure waveforms, to discriminate between normal and pathologic breaths. The new flow and airway pressure waveforms may or may not include a pathologic breath/breathing pattern. In some implementations, the non-transitory computer-readable medium of the waveform analysis platform 1506 further stores instructions for the spectral tensor technique 700 to generate the new spectral tensors 102. In other implementations, the waveform analysis platform 1506 receives the new spectral tensors 102 from another computing device. The system 1500 may generate an output/response 1508. The output 1508 may improve patient care and/or reduce the cost of patient care. For example, the output 1508 may be an annotation of the input waveform 1510. This may reduce the number of breaths that a clinician must examine and label. Thus, a clinician may be able to oversee the care of more patients. Annotation of the input waveform 1510 may be contemporaneous or retrospective (e.g., annotation of previously collected data). In one example, a waveform analysis platform 1506 with a PDB model 108, e.g., a binary DC breath detection model, operating at 92% sensitivity and 95% specificity reduces the amount of data that must be examined/reviewed by a clinician by 95%. As another example, the output 1508 may be a notification 1512 of a pathologic breath/breathing pattern. This may reduce the response time to provide the patient with appropriate care.

In at least one implementation, the waveform analysis platform 1506 includes a processor, a spectral tensor module configured to, when executed by the processor, generate a spectral tensor, and a PBD model 108 configured to, when executed by the processor, analyze a new waveform and identify a pathologic breath/pathologic breathing pattern in the new waveform. The waveform analysis platform 1506 may further include an annotation module configured to, when executed by the processor, identify and/or annotate the new waveform. For example, the annotation module may identify instances of inspiration and expiration. The output of the annotation module may be provided to the spectral tensor module and the output of the spectral tensor module may be provided to the PBD model 108.

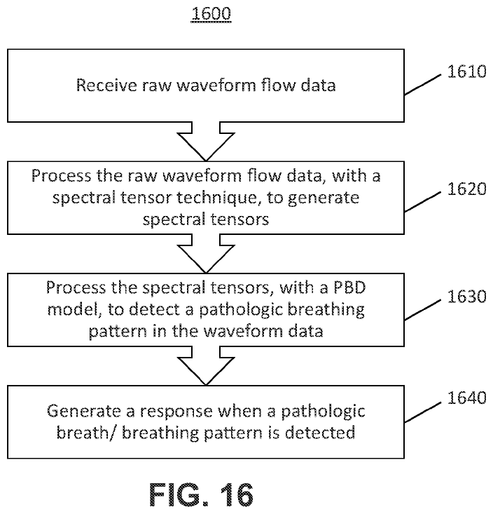

FIG. 16 is a flowchart of method to identify/diagnose a pathologic breath/breathing pattern that may be executed by the system 1500 illustrated in FIG. 15, according to some embodiments. At Step 1610 flow and airway pressure waveforms are received. At Step 1620, the waveforms are processed with a spectral tensor technique 700 to generate spectral tensors 102 of breath triplets in the waveform. The waveforms may be flow waveforms and/or airway pressure waveforms. In some implementations, the PBD model 108 may be used for a breath by breath analysis. For example, a breath triplet window may be shifted over the waveform to generate subsequent breath triplets across time—e.g., a current breath of a first breath triplet becomes a previous breath of a second breath triplet and a subsequent breath of the first breath triplet becomes a current breath of the second breath triplet. These sequential breath triplets are transformed into spectral tensors 102 by the spectral tensor technique 700.

At Step 1630, spectral tensors 102 generated from the waveform are processed/analyzed by the PBD model 108 to detect a pathologic breath/breathing pattern present in the waveforms. As discussed above, a fixed-length window 1310 may be applied to each spectrogram 1202, 1204 to produce a desired fixed-size image—a spectral image having the size of the window. In some implementations, the PBD model 108 processes the entire window of measurements simultaneously. For example, a PBD model 108 developed using a deep convolutional neural network (CNN) may process the entire window of measurements simultaneously.

At Step 1640 an output/response is generated when a pathologic breath/breathing pattern is detected. The response may be to annotate the waveform data and/or generate a notification. For example, the annotation module of the module waveform analysis platform 1506 may annotate the waveform data contemporaneously or retrospectively. The notification may include an alarm and/or a message. The message may be sent by text, email, or to pager. Step 1640 may reduce cost and/or improve patient care. For example, a PBD model 108 configured to annotate pathologic breaths/patterns on a waveform may reduce the number of breaths that a clinician must examine and label. As another example, a PBD model 108 configured to generate a notification may reduce the response time to provide the patient with appropriate care.

The steps of method 700 may be implemented as computer program code of a non-transitory computer readable medium that forms a part of a waveform analysis platform 1506. In some embodiments, method 700 is utilized in a method to annotate spirometer flow waveforms and/or airway pressure waveforms.

### Performance of PBD Models

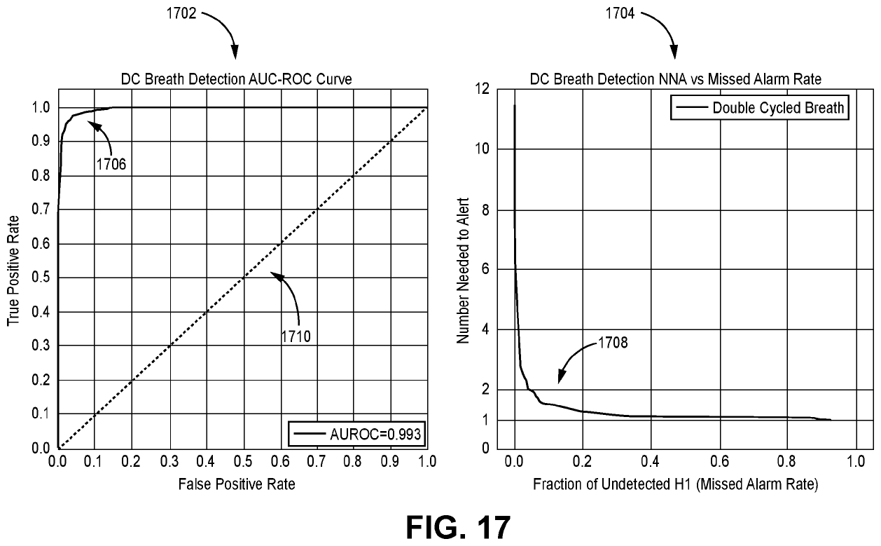

Graphs 1702, 1704 of FIG. 17 illustrate examples of the performance of a PDB model 108 that is a binary Double Cycle (DC) breath detection model. Graph 1702 illustrates a receiver operating characteristic curve 1706 and a random guess line 1710. As illustrated in graph 1702, the binary DC breath detection model 108 yielded an area under the receiver operating characteristic curve 1706 (AUROC) of 0.993. Graph 1704 illustrates the relationship between the number needed to alert (NNA) and the missed detection rate. In this example, when operating at 75.2% sensitivity, the PBD model 108 had an NNA of 1.2 (99.6% specificity), meaning that the binary Double Cycle (DC) breath detection model 108 had one false alarm for every five true alarms (line 1908). In the example, when operating at 90% sensitivity, the PBD model 108 had an NNA of 1.5 (98.7% specificity). In another example, a binary DC breath detection model 108, when operating at 75% sensitivity, had an NNA of 1.3 (99%) specificity—one false alarm for every three true alarms was observed—and a 91.8% specificity when operating at 90.2% sensitivity—equivalent to an NNA of 12.3 (not shown). A binary DC breath detection model 108 capable of operating at 75% sensitivity with minimal false positives (i.e., NNA=1.2) would be valuable for detecting DC breaths at the bedside.

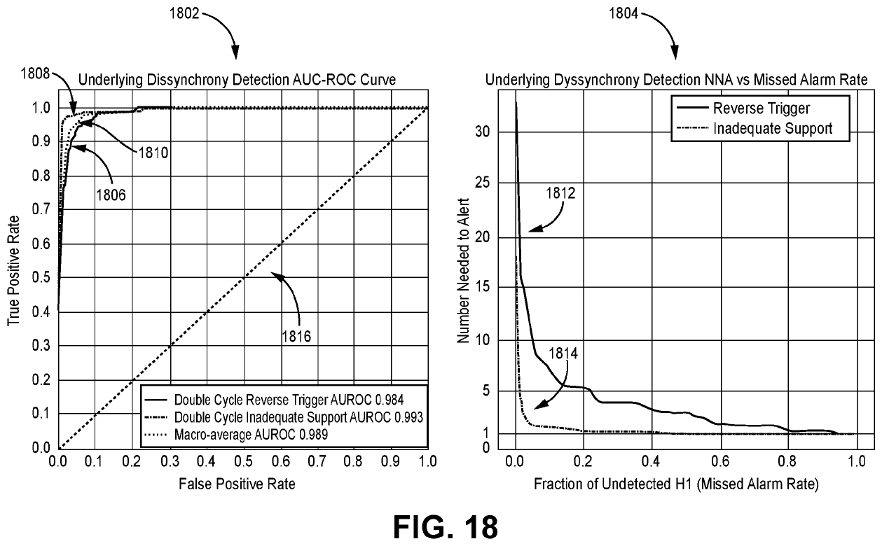

Graphs 1802, 1804 of FIG. 18 illustrate examples of the performance of a PBD model 108 that is a multi-target PBD model 108 for detecting Reverse Trigger and Inadequate Support as underlying dyssynchrony types. Graph 1802 illustrates receiver operating characteristic curves 1806, 1808, 1810 of multi-target PBD model 108 and a random guess line 1816. In this example, the multi-target underlying dyssynchrony detection model 108 yielded an AUROC of 0.984 (line 1806) when detecting Reverse Trigger, an AUROC of 0.993 (line 1808) when detecting Inadequate Support, and a macro-average AUROC of 0.989 (line 1810). Graph 1804 illustrates the relationship between the NNA and the missed detection rate of multi-target PBD model 108. In this example, then operating at a sensitivity of 75% for Reverse Trigger, the multi-target PBD model 108 had an NNA of 4 (98.4% specificity) (see line 1812), which indicates the multi-target PBD model 108 had three false alarms for every one true alarm. In this example, increasing Reverse Trigger sensitivity to 90.2% yielded 96.3% specificity. In this example, when operating at a sensitivity of 75.4% for Inadequate Support, the multi-target PBD model 108 had an NNA of 1.2 (99.7% specificity) (see line 1814), which indicates the multi-target PBD model 108 had one false alarm for every five true alarms. In this example, increasing Inadequate Support sensitivity to 90.2% resulted in 99.1% specificity. In another example, a multi-target PBD model 108, when operating at 75.1% sensitivity for Reverse Trigger, had an NNA of 1.6 (98.5% specificity)—three false alarms for every five true alarms—and a 93% specificity when operating at 90% sensitivity (not shown). In an additional example, a multi-target PBD model 108, when operating at a sensitivity of 75.2% for Inadequate Support had an NNA of 4 (98.2% specificity)—three false alarms for every one true alarm—and a 91.8 specificity when operating at a 90.2% sensitivity (not shown). The lower performance of the PBD model 108 in detecting Inadequate Support compared to detecting either DC breaths or Reverse Trigger is consistent with Cohen's kappa statistic for the annotations of 18,282 breaths by two clinicians. In some embodiments, two separate operating points may be selected for the multi-target PBD model 108. For example, the multi-target PBD model 108 may be operated at 92% sensitivity with 92% specificity for Reverse Trigger, and 95% sensitivity with 89% specificity for Inadequate Support. In one implementation, a multi-target PBD model 108 with two different operating points may be utilized for Reverse Trigger and Inadequate Support annotation.

The NNA values for the PBD models 108 discussed above indicate that, at reasonable detection thresholds, the models 108 have a manageable number of false alarms to prevent alarm fatigue with a reasonable level of sensitivity comparable to or better than other clinical alarm devices (e.g., continuous electrocardiogram monitors). Some non-limiting examples of PDB models 108 with low NNA and reasonable sensitivity include a binary DC breath detection model 108 with an NNA of 1.3 with 75% and a multi-target PBD model 108 operating at an NNA of 1.4 with 72% sensitivity for Reverse Trigger and NNA of 1.9 with 33% sensitivity for Inadequate Support.

As discussed above, utilizing both phase and PSD spectrograms as inputs to train the PDB model 104 may improve performance of the PDB model 108. For example, it was observed that binary DC breath detection models trained with phase and PSD spectrogram inputs, only phase spectrogram input, or only PSD spectrogram input, had AUROCs respectively of 0.984; 0.976, and 0.980. As another example, a multi-target PBD models trained with phase and PSD spectrogram inputs, only phase spectrogram input, or only PSD spectrogram input, were found to have AUROCs of 0.980; 0.961, and 0.945, respectively, when detecting Reverse Trigger, and AUROCs of 0.976, 0.861, and 0.931 respectively when detecting Inadequate Support.

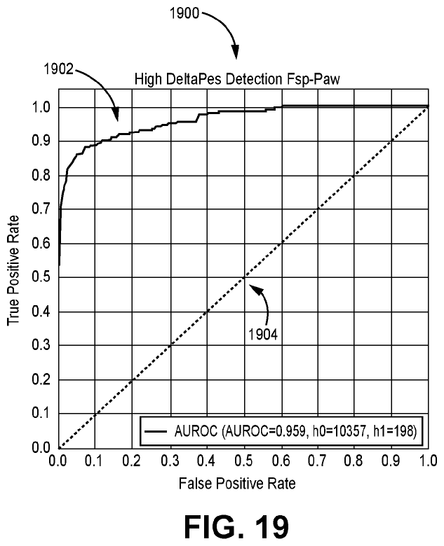

Graph 1900 of FIG. 19 illustrates the performance of a PBD model 108 that is a binary PBD model trained to detect high work of breathing. Graph 1900 illustrates a receiver operating characteristic curve 1902 and a random guess line 1904. In this non-limiting example, a high work of breathing is identified when ΔP_{ES}≥20 mmHg. The binary PBD model 108 for detecting high work of breathing achieved an AUROC of 0.959 on the validation set.

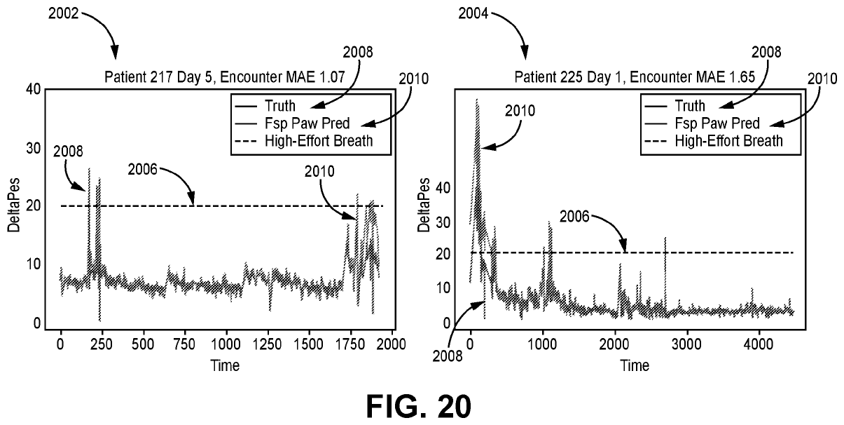

Graphs 2002 and 2004 of FIG. 20 illustrate the performance of a PBD model 108, a regression PBD model for predicting high respiratory effort, for two patients. Graphs 2002 and 2004 illustrate a comparison of true/actual values of ΔP_{ES} 2008 and predicted values 2010 generated by the regression PBD model 108. In this non-limiting example, a high work of breathing is identified when ΔP_{ES}≥20 mmHg. A horizontal red dashed line 2006 is drawn at 20 mmHg to indicate the threshold for high work of breathing. The regression PBD model 108 predicted the values of ΔP_{ES} with a mean absolute error (MAE) of 3 mmHg and a root mean squared error (RMSE) of 4 mmHg over all breaths in the validation set. When the predictions generated by the regression PBD model 108 were used to classify breaths as high work of breathing (ΔP_{ES}≥20 mmHg) or low (ΔP_{ES}<20 mmHg), the AUROC was 0.972.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto.

Various examples have been described. These and other examples are within the scope of the following claims.

1. A method comprising:
obtaining, by a computing device, a spectral tensor, wherein the spectral tensor is generated by:
generating a power spectrogram and a phase spectrogram for a breath triplet of a training waveform, wherein the training waveform is a flow waveform, an airway pressure waveform, and/or an esophageal manometry waveform;
removing high frequency bins from each spectrogram;
generating a spectral image by sizing each spectrogram to a pre-determined size;
assembling the spectral images generated for each breath triplet into the spectral tensor; and
training a machine learning model to detect a pathologic breath and/or pathologic breathing pattern in a waveform using the spectral tensor as a training input.

2. The method of claim 1, wherein the machine learning model is a convolutional neural network.

3. (canceled)

4. The method of claim 1 , wherein generating each spectrogram comprises applying a Fourier transform with a tapered cosine window of a predetermined size and at a predetermined stride between Fourier transforms to generate a plurality of spectral columns from the waveform.

5. The method of claim 4, wherein removing high frequency bins comprises applying a low pass filter.

6. The method of claim 1, wherein the breath triplet is generated by:
collecting the training waveform, each training waveform comprising a plurality of breaths;
identifying inspiration, expiration, asynchronies, artifacts and/or a respiratory effort in the training waveform; and
excluding a breath triplet with a breath having a time greater or less than a pre-selected time period and/or a breath triplet with a middle breath annotated as normal and a dyssynchronous left or right breath.

7. The method of claim 6, wherein identifying inspiration, expiration, asynchronies, artifacts and/or a respiratory effort in the training waveform includes:
annotating the training waveform; and/or
collating data about inspiration, expiration, asynchronies, artifacts and/or a respiratory effort into a dataset.

8. The method of claim 1, wherein spectral tensors generated from a single person are allocated to one of a training set, a validation set, and a test set.

9. A computer-implemented analysis method comprising:
obtaining, by the computer, a new waveform, the new waveform being either a flow waveform and/or an airway pressure waveform; and
evaluating the new waveform using a pathologic breath detection model to detect a pathologic breath and/or pathologic breathing pattern in the new waveform, wherein the pathologic breath detection model was trained using a spectral tensor as input, each spectral tensor generated from a breath triplet of a training waveform, wherein the training waveform is a flow waveform, an airway pressure waveform, and/or an esophageal manometry waveform, the spectral tensor generated by a spectral tensor technique comprising the steps of:
generating a power spectrogram and a phase spectrogram for the breath triplet;
removing high frequency bins from each spectrogram;
generating a spectral image by sizing each spectrogram to a pre-determined size; and
assembling the spectral images generated for each breath triplet into the spectral tensor.

10. The method of claim 9, wherein the breath triplet is generated by:
collecting the training waveform, each training waveform comprising a plurality of breaths;
identifying inspiration, expiration, asynchronies, artifacts and/or a respiratory effort in the training waveform; and
excluding a breath triplet with a breath having a time greater or less than a pre-selected time period and/or a breath triplet with a middle breath annotated as normal and a dyssynchronous left or right breath.

11. The method of claim 10, wherein identifying inspiration, expiration, asynchronies, artifacts and/or a respiratory effort in the training waveform includes:
annotating the training waveform; and/or
collating data about inspiration, expiration, asynchronies, artifacts and/or a respiratory effort into a dataset.

12. (canceled)

13. The method of claim 9, wherein the pathologic breath detection model comprises a convolutional neural network.

14. (canceled)

15. The method of claim 9, wherein generating each spectrogram comprises applying a Fourier transform with a tapered cosine window of a predetermined size and at a predetermined stride between Fourier transforms to generate a plurality of spectral columns from the waveform.

16. (canceled)

17. The method of claim 9, evaluating the new waveform further comprises utilizing the tensor technique to generate a spectral tensor for a breath triplet of the new waveform.

18. The method of claim 9, wherein
evaluating the new waveform further comprises:
generating sequential breath triplets for a breath by breath analysis of the new waveform; and
utilizing the tensor technique to generate a spectral tensor for each sequential breath triplet.

19. The method of claim 9, further comprising generating a response when a pathologic breath and/or pathologic breathing pattern is detected by the pathologic breath detection model.

20. The method of claim 9, wherein the pathologic breath detection model is:
a binary DC breath detection model;
a multi-target dyssynchrony detection model; or
a respiratory effort detection model.

21. (canceled)

22. A computer program product comprising a non-transitory computer readable medium have embodied thereon a computer program comprising computer code comprising:
code for a pathologic breath detection model to detect a pathologic breath and/or pathologic breathing pattern in a waveform, wherein the waveform is a flow waveform and/or an airway pressure waveform, wherein the pathologic breath detection model was trained with a spectral tensor generated by a method comprising:
generating a power spectrogram and a phase spectrogram for a breath triplet in a training waveform, wherein the training waveform is a flow waveform, an airway pressure waveform, and/or an esophageal manometry waveform;
removing high frequency bins from each spectrogram;
generating a spectral image by sizing each spectrogram to a pre-determined size; and
assembling the spectral images generated for each breath triplet into the spectral tensor.

23. The computer program product of claim 22, wherein the breath triplet is generated by:
collecting the training waveform, each training waveform comprises a plurality of breath;
identifying inspiration, expiration, asynchronies, artifacts and/or a respiratory effort in the training waveform; and
excluding a breath triplet with a breath having a time greater or less than a pre-selected time period and/or a breath triplet with a middle breath annotated as normal and a dyssynchronous left or right breath.

24-28. (canceled)

29. The computer program product of claim 22, wherein the pathologic breath detection model is:
a binary DC breath detection model;
a multi-target dyssynchrony detection model; or
a respiratory effort detection model.

30. The computer program product of claim 29,
wherein the respiratory effort detection model is a: binary respiratory effort detection model; or
a regression respiratory effort model.
